(19) United States
(12) Patent Application Publication
(10) Pub. No.: US 2023/0101982 A1
(43) Pub. Date: Mar. 30, 2023

(54) INITIAL CONFORMATION GENERATION APPARATUS, INITIAL CONFORMATION GENERATION METHOD, AND STORAGE MEDIUM

(71) Applicant: FUJITSU LIMITED, Kawasaki-shi (JP)

(72) Inventor: Yoshiaki TANIDA, Kawasaki (JP)

(73) Assignee: FUJITSU LIMITED, Kawasaki-shi (JP)

(21) Appl. No.: 17/825,151

(22) Filed: May 26, 2022

(30) Foreign Application Priority Data
Sep. 27, 2021 (JP) 2021-156817

(51) Int. Cl. G16B 15/00; G16B 30/00; G16C 20/50

(52) U.S. Cl. CPC G16B 15/00; G16B 30/00; G16C 20/50

(57) ABSTRACT

An initial conformation generation apparatus includes one or more memories; and one or more processors coupled to the one or more memories and the one or more processors configured to generate a model representing a cyclic peptide molecule by identifying Cα atoms of each of a plurality of amino acid residues, by arranging the identified Cα atoms on a circumference, and by adding main chains and side chains of the plurality of amino acid residues, and search for a stable conformation of the cyclic peptide molecule by using the generated model as an initial conformation of the cyclic peptide molecule.

### CROSS-REFERENCE TO RELATED APPLICATION

This application is based upon and claims the benefit of priority of the prior Japanese Patent Application No. 2021-156817, filed on Sep. 27, 2021, the entire contents of which are incorporated herein by reference.

### FIELD

The present disclosure relates to an initial conformation generation apparatus, an initial conformation generation method, and a storage medium.

### BACKGROUND

In recent years, in the field of drug discovery, drug discovery based on middle molecules (molecular weight of 500 to 3000) supposed to cause less side effects has been expected, and development of a search method for searching for a stable conformation of a middle molecule has been in progress.

For example, a cyclic peptide molecule has been drawing great attention because cyclization greatly reduces the entropy, which makes it possible to generate a compound having great binding activity.

Japanese Laid-open Patent Publication Nos. 2010-159218 and 2020-091518 and U.S. Patent Application Publication No. 2018/0260517 are disclosed as related art.

Also, F. Jiang and H. Geng, “Computational Methods for Studying Conformational Behaviors of Cyclic Peptides”, 2019 is disclosed as the related art.

### SUMMARY

According to an aspect of the embodiments, an initial conformation generation apparatus includes one or more memories; and one or more processors coupled to the one or more memories and the one or more processors configured to generate a model representing a cyclic peptide molecule by identifying Cα atoms of each of a plurality of amino acid residues, by arranging the identified Cα atoms on a circumference, and by adding main chains and side chains of the plurality of amino acid residues, and search for a stable conformation of the cyclic peptide molecule by using the generated model as an initial conformation of the cyclic peptide molecule.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of a system configuration of a stable conformation search system and functional configurations of a terminal apparatus and a server apparatus;

FIG. 2A and FIG. 2B are diagrams illustrating an example of hardware configurations of the terminal apparatus and the server apparatus;

FIG. 3 is a diagram illustrating an example of a functional configuration of a cyclic peptide molecule generation unit;

FIG. 4 is a diagram illustrating a specific example of processing by a Cα atom arrangement unit;

FIG. 5 is a diagram illustrating a specific example of processing by an addition unit and a conformational relaxation unit;

FIG. 6 is a diagram for explaining backbone dihedral angles;

FIG. 7 is a first diagram illustrating an example of a dihedral angle distribution diagram;

FIG. 8 is a second diagram illustrating an example of a dihedral angle distribution diagram; and

FIG. 9 is a flowchart illustrating a sequence of stable conformation search processing.

### DESCRIPTION OF EMBODIMENTS

In searching for a stable conformation, it is important to search an appropriate search range, and whether the search range is optimized or not largely depends on an initial conformation.

Meanwhile, in the case of a cyclic peptide molecule, the initial conformation has been generated by a method of cyclizing a linear peptide molecule by linking a head and a tail thereof. For this reason, in the generated initial conformation, each of amino acid residue sequences constituting the cyclic peptide molecule has such low degrees of freedom that an appropriate search range may not be searched in searching for a stable conformation.

According to one aspect, an object is to optimize a search range in searching for a stable conformation of a cyclic peptide molecule.

### Advantageous Effects of Invention

A search range in searching for a stable conformation of a cyclic peptide molecule may be optimized.

Hereinafter, the embodiments will be described with reference to the accompanying drawings. In the present specification and drawings, constituent elements having substantially the same functional configuration will be denoted with the same reference sign, whereby repetitive description thereof will be omitted.

### First Embodiment

<System Configuration of Stable Conformation Search System and Functional Configurations of Terminal Apparatus and Server Apparatus>

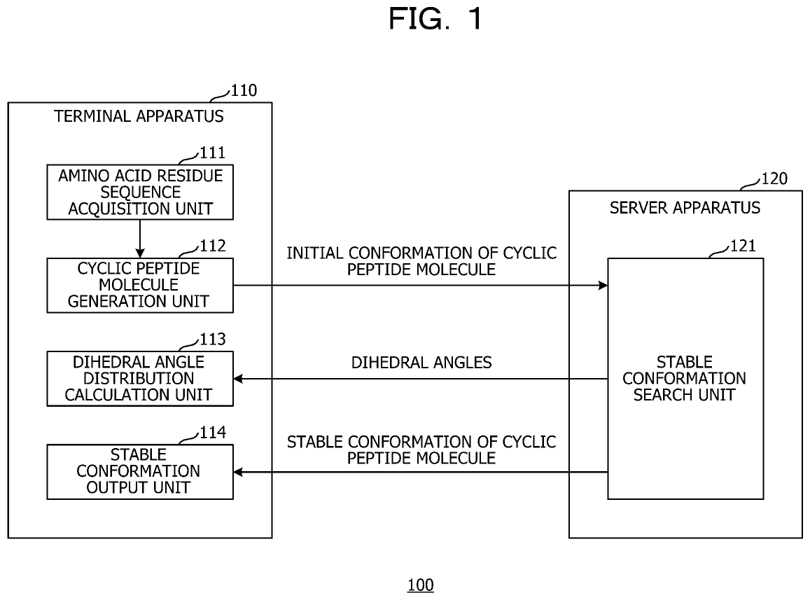

First, description will be given of a system configuration of a stable conformation search system according to a first embodiment and functional configurations of a terminal apparatus and a server apparatus constituting the stable conformation search system. FIG. 1 is a diagram illustrating an example of the system configuration of the stable conformation search system and the functional configurations of the terminal apparatus and the server apparatus.

A stable conformation search system 100 is a system for searching for a stable conformation of a middle molecule and, for example, a system for searching for a stable conformation of a cyclic peptide molecule having multiple amino acid residue sequences.

As illustrated in FIG. 1, the stable conformation search system 100 includes a terminal apparatus 110, which is an example of an initial conformation generation apparatus, and a server apparatus 120.

A search program is installed in the terminal apparatus 110. Executing the program, the terminal apparatus 110 functions as an amino acid residue sequence acquisition unit 111, a cyclic peptide molecule generation unit 112, a dihedral angle distribution calculation unit 113, and a stable conformation output unit 114.

The amino acid residue sequence acquisition unit 111 is an example of an acquisition unit, and acquires multiple amino acid residue sequences constituting a cyclic peptide molecule of a search target when a user of the stable conformation search system 100 inputs these amino acid residue sequences.

When the user of the stable conformation search system 100 inputs a parameter (a distance “a” to be described later) for use to generate an initial conformation of the cyclic peptide molecule of the search target, the amino acid residue sequence acquisition unit 111 acquires the parameter.

The cyclic peptide molecule generation unit 112 is an example of a generation unit, and generates, as the initial conformation of the cyclic peptide molecule, a model representing the cyclic peptide molecule under the acquired parameter based on the multiple amino acid residue sequences acquired by the amino acid residue sequence acquisition unit 111. The cyclic peptide molecule generation unit 112 is also an example of a search instruction unit, and transmits information indicating the generated initial conformation of the cyclic peptide molecule to the server apparatus 120, thereby instructing the server apparatus 120 to search for a stable conformation based on the initial conformation. Details of a method for generating the initial conformation of the cyclic peptide molecule by the cyclic peptide molecule generation unit 112 will be described later.

The dihedral angle distribution calculation unit 113 is an example of a calculation unit. In response to transmission of the information indicating the initial conformation of the cyclic peptide molecule by the cyclic peptide molecule generation unit 112, the dihedral angle distribution calculation unit 113 acquires information indicating backbone dihedral angles calculated in the process of searching for the stable conformation from the server apparatus 120. The dihedral angle distribution calculation unit 113 calculates a dihedral angle distribution based on the acquired information indicating the backbone dihedral angles, and outputs a dihedral angle distribution diagram.

In response to the transmission of the information indicating the initial conformation of the cyclic peptide molecule by the cyclic peptide molecule generation unit 112, the stable conformation output unit 114 acquires information indicating the searched-out stable conformation of the cyclic peptide molecule from the server apparatus 120. The stable conformation output unit 114 outputs the acquired information indicating the stable conformation of the cyclic peptide molecule.

The server apparatus 120 functions as a stable conformation search unit 121. The stable conformation search unit 121 is an example of a search unit. When receiving the information indicating the initial conformation of the cyclic peptide molecule from the terminal apparatus 110, the stable conformation search unit 121 searches for a stable conformation of the cyclic peptide molecule based on the received information. The stable conformation search unit 121 transmits information indicating the backbone dihedral angles calculated in the process of searching for the stable conformation, to the terminal apparatus 110. The stable conformation search unit 121 transmits the information indicating the searched-out stable conformation of the cyclic peptide molecule to the terminal apparatus 110.

Based on the information indicating the initial conformation of the cyclic peptide molecule transmitted from the terminal apparatus 110, the stable conformation search unit 121 searches for the stable conformation by, for example, a generalized-ensemble method. Examples of the generalized-ensemble method mentioned herein include a multi-canonical method, a simulated tempering method, a replica exchange method (for example, replica exchange with solute tempering (REST) 2), and so on.

<Hardware Configurations of Terminal Apparatus and Server Apparatus>

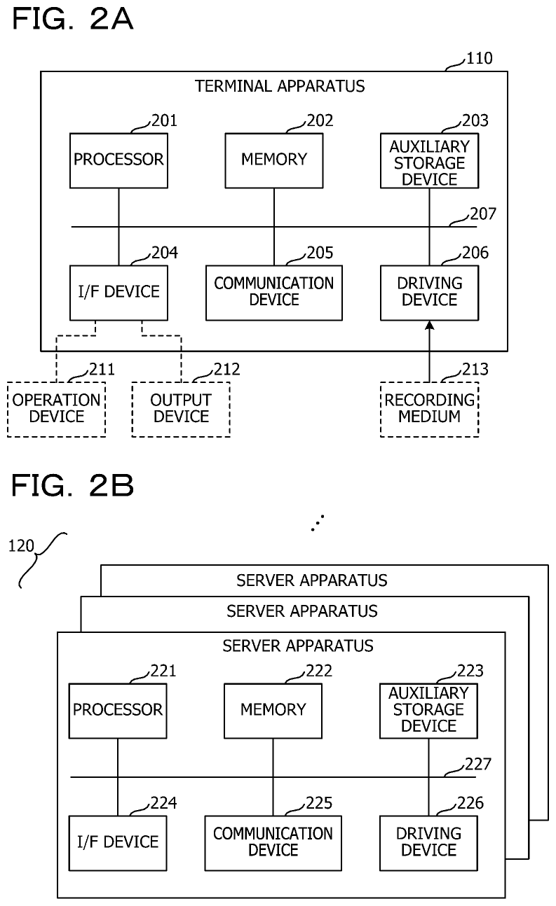

Next, hardware configurations of the terminal apparatus 110 and the server apparatus 120 will be described next. FIG. 2A and FIG. 2B are diagrams illustrating an example of the hardware configurations of the terminal apparatus and the server apparatus.

(1) Hardware Configuration of Terminal Apparatus

As illustrated in FIG. 2A, the terminal apparatus 110 includes a processor 201, a memory 202, an auxiliary storage device 203, an interface (I/F) device 204, a communication device 205, and a driving device 206. These hardware devices in the terminal apparatus 110 are coupled to each other via a bus 207.

The processor 201 includes various computing devices such as a central processing unit (CPU) and a graphics processing unit (GPU). The processor 201 loads various programs (such, for example, as a search program) onto the memory 202 and executes the programs.

The memory 202 includes main storage devices such as a read-only memory (ROM) and a random-access memory (RAM). The processor 201 and the memory 202 form a so-called computer. The computer implements the above various functions by the processor 201 executing the various programs loaded onto the memory 202.

The auxiliary storage device 203 stores the various programs and various kinds of information to be used in execution of the various programs by the processor 201.

The I/F device 204 is a coupling device that couples the terminal apparatus 110 to an operation device 211 and an output device 212, which are examples of external devices.

The communication device 205 is a communication device for communicating with the server apparatus 120 via a network.

The driving device 206 is a device in which a recording medium 213 is to be placed. Examples of the recording medium 213 mentioned herein include a medium on which information is recorded optically, electrically, or magnetically, such as a compact disc read-only memory (CD-ROM), a flexible disk, and a magneto-optical disk. The examples of the recording medium 213 may also include a semiconductor memory and the like on which information is recorded electrically, such as a ROM and a flash memory.

The various programs to be installed onto the auxiliary storage device 203 are installed, for example, in such a way that the distributed recording medium 213 is placed in the driving device 206 and the driving device 206 reads out the various programs recorded on the recording medium 213. Alternatively, the various programs to be installed onto the auxiliary storage device 203 may be installed by being downloaded from the network via the communication device 205.

(2) Hardware Configuration of Server Apparatus

As illustrated in FIG. 2B, multiple server apparatuses 120 constitute a cluster, and each server apparatus includes a processor 221, a memory 222, an auxiliary storage device 223, an interface (I/F) device 224, a communication device 225, and a driving device 226.

The hardware devices included in the server apparatuses are the same as or similar to the hardware devices included in the terminal apparatus 110, and thus description thereof is omitted herein.

<Functional Configuration of Cyclic Peptide Molecule Generation Unit>

Next, a functional configuration of the cyclic peptide molecule generation unit 112 will be described in detail. As described above, executing the search program, the terminal apparatus 110 implements the various functions of the terminal apparatus 110. Among them, the cyclic peptide molecule generation unit 112 is implemented by executing, for example, an initial conformation generation program which is a part of the search program.

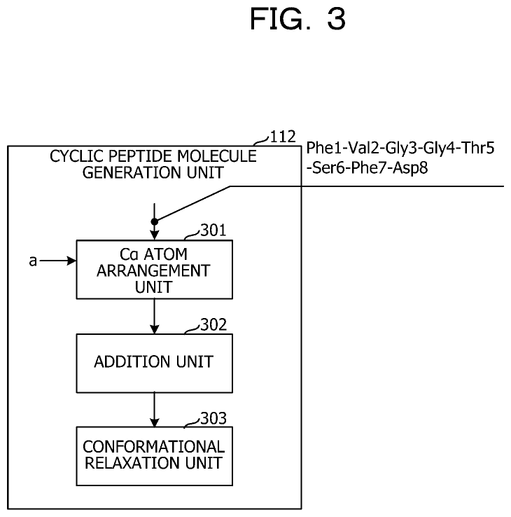

FIG. 3 is a diagram illustrating an example of the functional configuration of the cyclic peptide molecule generation unit. As illustrated in FIG. 3, the cyclic peptide molecule generation unit 112 includes a Cα atom arrangement unit 301, an addition unit 302, and a conformational relaxation unit 303.

The Cα atom arrangement unit 301 identifies a Cα atom that is an atom representing an amino acid in each of the acquired multiple amino acid residue sequences, and arranges the identified multiple Cα atoms at equal intervals on a circumference in the same plane. FIG. 3 illustrates the example in which 8 amino acid residue sequences (Phe1, Val2, Gly3, Thr5, Ser6, Phe7, and Asp8) are acquired and input to the Cα atom arrangement unit 301. The Cα atoms are arranged at equal intervals on the circumference in the same plane in order to constitute a conformation in which the multiple amino acid residue sequences (here, the 8 amino acid residue sequences) have higher degrees of freedom from each other.

When receiving a distance “a” (for example, 3.84 angstrom) between adjacent Cα atoms input by the user, the Cα atom arrangement unit 301 calculates a radius of a circumference where to arrange the Cα atoms based on the number “n” of the Cα atoms identified and the input distance “a” between the Cα atoms.

The Cα atom arrangement unit 301 arranges the Cα atoms at equal intervals along the circumference defined by the calculated radius such that the distance between the adjacent Cα atoms is “a”. The equal intervals mentioned herein do not have to be exactly at the same distance, but are synonymous with approximately equal intervals.

The addition unit 302 adds the main chain and side chains of each of the amino acid residue sequences to the corresponding one of the Cα atoms arranged on the circumference in the same plane.

The conformational relaxation unit 303 sequentially performs conformational relaxation of each of the amino acid residue sequences generated by adding the main chain and the side chains of each of the amino acid residue sequence to the corresponding one of the Cα atoms, thereby generating a model representing the cyclic peptide molecule having the multiple amino acid residue sequences as an initial conformation of the cyclic peptide molecule.

<Specific Example of Processing by Cα Atom Arrangement Unit>

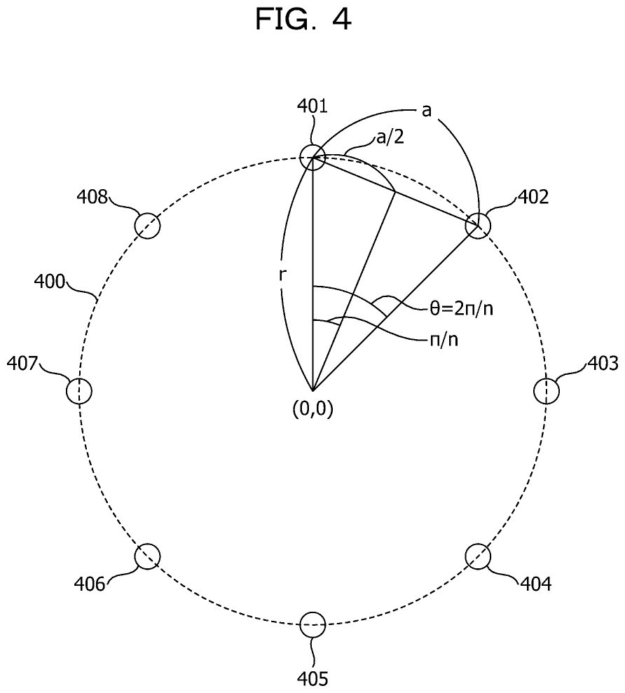

Next, a specific example of processing by the Cα atom arrangement unit 301 will be described. FIG. 4 is a diagram illustrating the specific example of the processing by the Cα atom arrangement unit. FIG. 4 illustrates the example in which Cα atoms 401 to 408 are identified respectively in n=8 amino acid residue sequences and arranged at equal intervals on a circumference 400 in the same plane.

As illustrated in FIG. 4, in the case of the 8 Cα atoms, among straight lines extending from the center coordinates (0, 0) of the circumference 400 to the respective Cα atoms, the straight lines extending to adjacent Cα atoms form an angle θ of 2π/n (=π/4).

As illustrated in FIG. 4, letting “a” be the distance between adjacent Cα atoms, a radius r of the circumference 400 may be calculated in accordance with the following formula (1):

r=(a/2)×[sin(π/n)]⁻¹. (Formula 1)

The coordinates of each of the Cα atoms 401 to 408 may be calculated in accordance with the following formula (2):

Coordinates=[r sin(n−1)θₓ,r cos(n−1)θₓ]. (Formula 2)

In the above formula (2), r denotes the radius of the circumference 400 calculated by the formula (1), and θₓ may be calculated in accordance with the following formula (3):

θₓ=x×2π/n, (Formula 3)

where x is any integer of 1 to n.

<Specific Example of Processing by Addition Unit and Conformational Relaxation Unit>

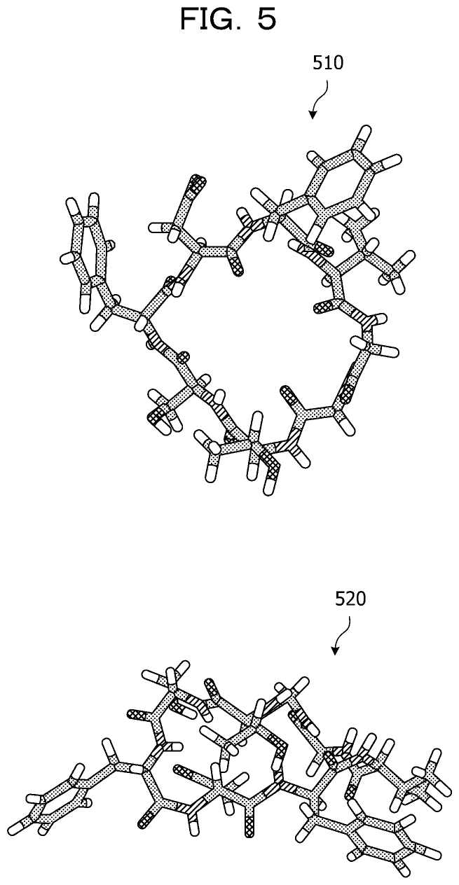

Next, a specific example of processing by the addition unit 302 and the conformational relaxation unit 303 will be described. FIG. 5 is a diagram illustrating the specific example of the processing by the addition unit and the conformational relaxation unit.

In FIG. 5, reference sign 510 illustrates an addition result in which the addition unit 302 adds the main chain and the side chains of each of the amino acid residue sequences to the corresponding one of the Cα atoms 401 to 408 arranged at the equal intervals on the circumference in the same plane by the Cα atom arrangement unit 301. When acquiring the addition result illustrated with reference sign 510 from the addition unit 302, the conformational relaxation unit 303 relaxes the post-addition conformation so as to reduce the entropy, and thereby generates a model representing the cyclic peptide molecule as an initial conformation of the cyclic peptide molecule.

Reference sign 520 illustrates a case where the initial conformation of the cyclic peptide molecule is generated in accordance with the method in the related art. The method in the related art is a method in which a linear peptide molecule is generated by linking 8 amino acid residue sequences in a linear chain form, and then cyclized by linking the head and the tail of the generated linear peptide molecule.

As is apparent from the comparison between reference signs 510 and 520, the initial conformation illustrated with reference sign 510 is a conformation in which the 8 amino acid residue sequences are farther from each other and therefore have higher degrees of freedom than in the initial conformation illustrated with reference sign 520.

<Specific Example of Processing by Dihedral Angle Distribution Calculation Unit>

Next, a specific example of processing by the dihedral angle distribution calculation unit 113 will be described. As described above, the dihedral angle distribution calculation unit 113 acquires the information indicating the backbone dihedral angles calculated in the process of searching for the stable conformation from the server apparatus 120, calculates the dihedral angle distribution based on the acquired information indicating the dihedral angles, and outputs the dihedral angle distribution diagram.

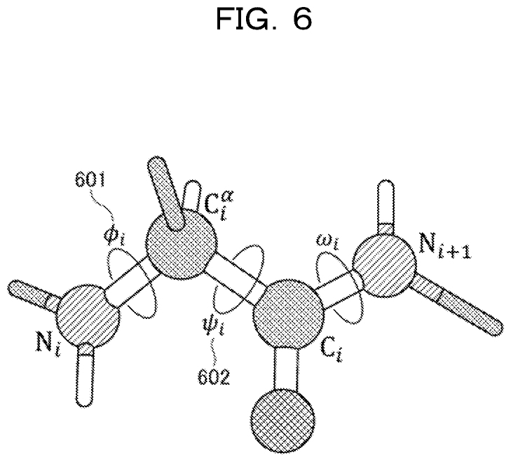

The backbone dihedral angles will be briefly described herein. FIG. 6 is a diagram for explaining the backbones dihedral angle. In the present embodiment, the dihedral angle distribution calculation unit 113 acquires (φ, ψ) as the information indicating the dihedral angles from the server apparatus 120. In FIG. 6, reference signs 601 and 602 respectively point to φᵢ and ψᵢ which are information indicating an i-th dihedral angle in the information indicating the dihedral angles acquired by the dihedral angle distribution calculation unit 113.

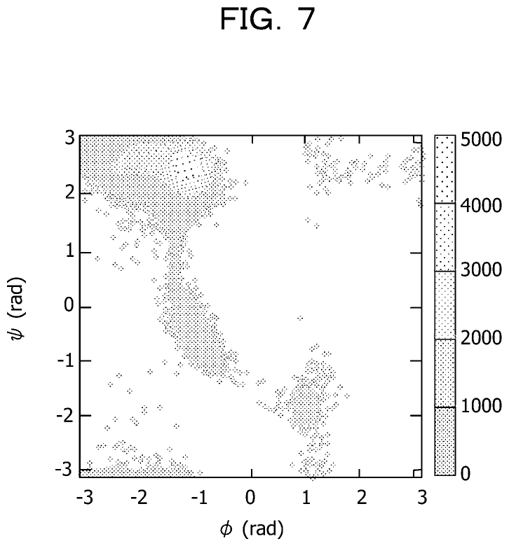

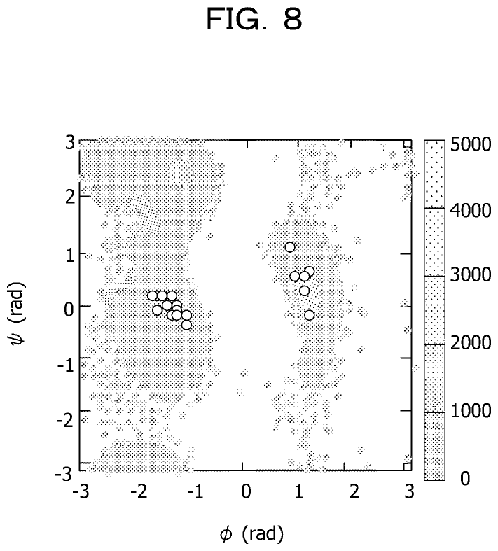

Subsequently, a dihedral angle distribution diagram will be described. FIGS. 7 and 8 are first and second diagrams illustrating examples of dihedral angle distribution diagrams. In FIGS. 7 and 8, the horizontal axis indicates φ [rad] in the information indicating the dihedral angles, and the vertical axis indicates ψ [rad] in the information indicating the dihedral angles. Each plot (Ramachandran plot) in the figures indicates that information indicating a dihedral angle corresponding to each position is calculated in the process of searching for a stable conformation, and a difference in color in the plot represents a difference in the number of searches performed on the information.

For example, the closer to red, the larger the number of searches is, and the closer to blue, the smaller the number of searches is. A white region indicates that the region is not searched even once.

FIG. 7 is a dihedral angle distribution diagram of Ser6 in a case where an initial conformation for 8 amino acid residue sequences (Phe1, Val2, Gly3, Thr5, Ser6, Phe7, and Asp8) is generated by the method in the related art and then a stable conformation is searched for. As described above, the method in the related art is the method in which a linear peptide molecule is generated by linking 8 amino acid residue sequences in a linear chain form, and is cyclized by linking the head and the tail of the generated linear peptide molecule.

FIG. 8 is a dihedral angle distribution diagram of Ser6 in a case where an initial conformation for 8 amino acid residue sequences (Phe1, Val2, Gly3, Thr5, Ser6, Phe7, and Asp8) is generated by the cyclic peptide molecule generation unit 112 and then a stable conformation is searched for.

As seen from the comparison between FIGS. 7 and 8, a difference between the initial conformations results in a large difference between the dihedral angle distribution diagrams. For example, in the search for a stable conformation by the server apparatus 120, the search range significantly differs between the different initial conformations.

In the present embodiment, which of the search ranges is more appropriate is examined by the following procedure: acquiring 20 kinds of stable conformations obtained by a nuclear magnetic resonance experiment for Ser6 among the above 8 amino acid residue sequences (Phe1, Val2, Gly3, Thr5, Ser6, Phe7, and Asp8); plotting, as correct answer data, the information indicating the dihedral angles in each of the 20 kinds of stable conformations acquired for Ser6 over a dihedral angle distribution diagram; and examining whether the correct answer data is included in the search range indicated by the plot of each color in the dihedral angle distribution diagram by checking a degree of overlap between the search range and the white circles plotted as the correct answer data.

In FIG. 8, 20 white circles represent the information indicating the dihedral angles in the 20 kinds of stable conformations acquired for Ser6. As illustrated in FIG. 8, all the white circles are located on the plot of blue to light blue, and are searched at least once by the stable conformation search unit 121 in the search for the stable conformation.

When the 20 white circles illustrated in FIG. 8 are plotted in the same positions over the dihedral angle distribution diagram illustrated in FIG. 7, some of the 20 white circles are plotted in the white region. For example, when the initial conformation of the cyclic peptide molecule is generated by the method in the related art, some of the 20 kinds of stable conformations are not searched even once in the search for the stable conformation.

For example, in a case where the initial conformation of the cyclic peptide molecule is generated by operating the cyclic peptide molecule generation unit 112, it is possible to search stable conformations that are not searched in a case where the initial conformation is generated by the method in the related art. For example, in the case where the initial conformation of the cyclic peptide molecule is generated by operating the cyclic peptide molecule generation unit 112, the server apparatus 120 is enabled to search a more appropriate search range than in the case where the initial conformation is generated by the method in the related art.

<Sequence of Stable Conformation Search Processing>

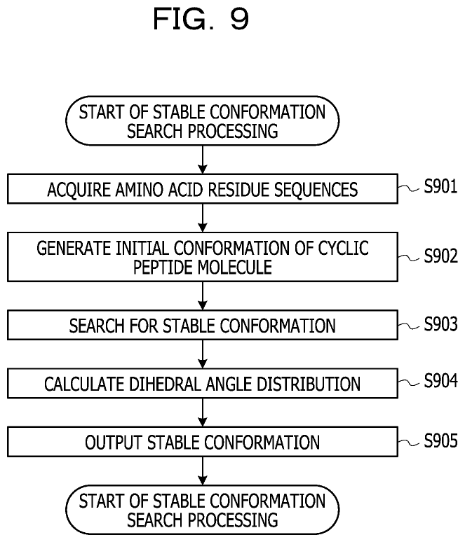

Next, a sequence of entire stable conformation search processing will be described. FIG. 9 is a flowchart illustrating the sequence of the stable conformation search processing.

At step S901, the terminal apparatus 110 acquires multiple amino acid residue sequences. The terminal apparatus 110 acquires a distance a between adjacent Cα atoms.

At step S902, the terminal apparatus 110 identifies the Cα atom of each of the acquired multiple amino acid residue sequences, and arranges the Cα atoms at equal intervals on the circumference in the same plane calculated based on the distance a. The terminal apparatus 110 adds the main chain and side chains of each of the amino acid residue sequence to the corresponding one of the arranged Cα atoms, and relaxes the post-addition conformation, thereby generating the initial conformation of the cyclic peptide molecule.

At step S903, the terminal apparatus 110 transmits information indicating the generated initial conformation of the cyclic peptide molecule to the server apparatus 120, thereby giving an instruction to search for a stable conformation of the cyclic peptide molecule.

At step S904, the terminal apparatus 110 acquires the information indicating the backbone dihedral angles calculated in the process of searching for the stable conformation from the server apparatus 120, calculates the dihedral angle distribution based on the acquired information indicating the dihedral angles, and outputs the dihedral angle distribution diagram to the user.

At step S905, the terminal apparatus 110 acquires the information indicating the searched-out stable conformation of the cyclic peptide molecule from the server apparatus 120, and outputs the information to the user.

As is clear from the above description, the terminal apparatus 110 according to the first embodiment identifies the Cα atom of each of the multiple amino acid residue sequences, arranges the Cα atoms at the equal intervals on the circumference, and adds the main chain and the side chains of each of the multiple amino acid residue sequences to the corresponding one of the Cα atoms arranged on the circumference. In this way, the terminal apparatus 110 according to the first embodiment is capable of generating the initial conformation of the cyclic peptide molecule in which each of the multiple amino acid residue sequences has higher conformational degrees of freedom.

The terminal apparatus 110 according to the first embodiment transmits the information indicating the generated initial conformation of the cyclic peptide molecule to the server apparatus 120, thereby giving the instruction to search for the stable conformation of the cyclic peptide molecule.

According to the first embodiment, it is thus possible to optimize a search range in a search for a stable conformation of a cyclic peptide molecule.

### Second Embodiment

The first embodiment is described above for the case where the stable conformation search system 100 is constituted by the terminal apparatus 110 and the server apparatuses 120. However, the stable conformation search system 100 may be constituted by the terminal apparatus 110, the server apparatus 120, and another apparatus (for example, three or more apparatuses). Alternatively, the stable conformation search system 100 may be constituted by an apparatus in which the terminal apparatus 110 and the server apparatus 120 are integrated (for example, a single apparatus).

The first embodiment is described above for the case where the terminal apparatus 110 includes the amino acid residue sequence acquisition unit 111 to the stable conformation output unit 114, and the server apparatus 120 includes the stable conformation search unit 121. However, some functions of the terminal apparatus 110 may be implemented in the server apparatus 120. Alternatively, some functions of the server apparatus 120 may be implemented in the terminal apparatus 110.

Although the first embodiment is described above for the example in which the 8 amino acid residue sequences are used to generate the initial conformation of the cyclic peptide molecule, the number of amino acid residue sequences for generating the initial conformation of the cyclic peptide molecule may be any number. Although the first embodiment is described above for the case where the dihedral angle distribution diagram for Ser6 among the 8 amino acid residue sequences is output, the dihedral angle distribution diagrams for the remaining 7 amino acid residue sequences may be output together.

Although details of the conformational relaxation are not described in the first embodiment, the conformational relaxation unit 303 may relax the conformation while keeping each Cα atom fixed, for example. Alternatively, the conformational relaxation unit 303 may relax the conformation including each Cα atom. Instead, the search for the stable conformation may be started without the conformational relaxation by the conformational relaxation unit 303 being executed.

The present disclosure is not limited to the configurations illustrated herein but may include configurations such as a combination of any of the configurations exemplified in the aforementioned embodiments with other elements. These aspects may be changed without departing from the gist of the present disclosure and appropriately set in accordance with application modes thereof.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. An initial conformation generation apparatus comprising:
one or more memories; and
one or more processors coupled to the one or more memories and the one or more processors configured to
generate a model representing a cyclic peptide molecule by identifying Cα atoms of each of a plurality of amino acid residues, by arranging the identified Cα atoms on a circumference, and by adding main chains and side chains of the plurality of amino acid residues, and
search for a stable conformation of the cyclic peptide molecule by using the generated model as an initial conformation of the cyclic peptide molecule.

2. The initial conformation generation apparatus according to claim 1, wherein the one or more processors are further configured to
arrange the Cα atoms at equal intervals on the circumference.

3. The initial conformation generation apparatus according to claim 1, wherein the one or more processors are further configured to
arrange each of n Cα atoms at a position of coordinates=[r sin(n−1)θₓ, r cos(n−1)θₓ], a distance between adjacent Cα atoms being a, r being (a/2)×[sin (π/n)]⁻¹, θₓ being x×2π/n, and x being an integer of 1 to n.

4. The initial conformation generation apparatus according to claim 1, wherein the one or more processors are further configured to
generate the model representing the cyclic peptide molecule by rearranging a conformation after the adding.

5. The initial conformation generation apparatus according to claim 4, wherein the one or more processors are further configured to
rearrange the conformation after the adding with keeping arrangement of the Cα atoms fixed.

6. The initial conformation generation apparatus according to claim 1,
wherein the one or more processors are further configured to acquire a dihedral angle distribution based on backbone dihedral angles acquired in the causing.

7. The initial conformation generation apparatus according to claim 6, wherein the one or more processors are further configured to
output the dihedral angle distribution in colors each corresponding to the number of searches for the stable conformation performed.

8. The initial conformation generation apparatus according to claim 1,
wherein the one or more processors are further configured to search for the stable conformation by a generalized-ensemble method using the generated model as the initial conformation of the cyclic peptide molecule.

9. An initial conformation generation method for a computer to execute a process comprising:
generating a model representing a cyclic peptide molecule by identifying Cα atoms of each of a plurality of amino acid residues, by arranging the identified Cα atoms on a circumference, and by adding main chains and side chains of the plurality of amino acid residues; and
searching for a stable conformation of the cyclic peptide molecule by using the generated model as an initial conformation of the cyclic peptide molecule.

10. A non-transitory computer-readable storage medium storing an initial conformation generation program that causes at least one computer to execute a process, the process comprising:
generating a model representing a cyclic peptide molecule by identifying Cα atoms of each of a plurality of amino acid residues, by arranging the identified Cα atoms on a circumference, and by adding main chains and side chains of the plurality of amino acid residues; and
searching for a stable conformation of the cyclic peptide molecule by using the generated model as an initial conformation of the cyclic peptide molecule.
